(19) United States
(12) Patent Application Publication
(10) Pub. No.: US 2003/0223934 A1
(43) Pub. Date: Dec. 4, 2003

(54) Electrostatically charged nasal application diagnotic product and method

(75) Inventor: Ashok L. Wahi, Hillsborough, NJ (US)

Correspondence Address: KENNETH P. GLYNN, ESQ., Glynn & Associates, P.C., 24 Mine Street, Flemington, NJ, 08822, US

(73) Assignee: Trutek Corp

(21) Appl. No.: 10/161,821

(22) Filed: Jun. 4, 2002

(51) Int. Cl. A61K 49/00

(52) U.S. Cl. 424/9.1

(57) ABSTRACT

Disclosed is a diagnostic method for determining the presence or absence of airborne contaminants. This involves applying a nasal topical application product for restricting the flow of airborne contaminants near human nasal passages to create a proximate and enhanced electrostatic field. This attracts and holds the airborne contaminant for subsequent presentation to known diagnostics testing. The nasal application product includes: (a) a plurality of masses of one or more electrostatic polymers; (b) a topical carrier having the plurality of masses dispersed through a portion thereof. The nasal application product may be topical solutions, semisolids, spray solutions and vaporizable solutions. Topical applications may be in the form of ointments, pastes, creams and gels. The carrier of the nasal application product of the present invention may be selected from the group consisting of diluents, volatile spray carriers, lotions, solvents, gels and hydro-gels. In some embodiments, substrates, e.g., bandage type substrates, with adhesive on one side and the product polymer(s) and carrier on the opposite side, may be employed. In other embodiments a fluid, which when dried to film is removable by peeling for testing, may by utilized.

### I. DESCRIPTION

### A. INCORPORATIONS BY REFERENCE

This application is related to U.S. Pat. No. 5,468,488, entitled “ELECTROSTATICALLY CHARGED NASAL APPLICATION PRODUCT AND METHOD” issued to Ashok L. Wahi, inventor, on Nov. 21, 1995.

Secondly, this application is additionally related to U.S. Pat. No. 5,674,481, entitled “ELECTROSTATICALLY CHARGED NASAL APPLICATION PRODUCT”, also issued to Ashok L. Wahi on Oct. 7, 1997.

Thirdly, this application is furthermore related to United States pending patent application, entitled “ELECTROSTATICALLY CHARGED NASAL APPLICATION PRODUCT WITH INCREASED STRENGTH”, United States application serial number 10/082,978 filed on Feb. 25, 2002, by Ashok L. Wahi, the inventor herein.

### B. TECHNICAL FIELD OF THE INVENTION

The present invention relates to diagnostic methods that involve the use of products that were heretofore developed for restricting the flow of airborne contaminants into the nasal passages by creating an electrostatic field in an area near about the nasal passages. This reduced the inflow of airborne contaminants to the nasal passages. In the present invention, these electrostatically charged nasal application products are used to collect airborne contaminants for subsequent diagnostic testing to determine whether the user was exposed to airborne contaminant(s) and establish the nature, type, and specificities of such contaminant(s).

### II. BACKGROUND OF THE INVENTION

A. Field of the Invention

A number of diagnostic tests may be performed on humans or their environments to evaluate the existence and amount of contaminants present in the ambient air. The diagnostic tests typically check organic and/or inorganic particulate that may be either innocuous or harmful to the user.

Allergies are a major health concern. Treatment of allergic condition(s) may include avoidance, dust mite control, pollen control, medication, immuno-therapy, enzyme therapy, acupuncture/acupressure, chiropractic care, reflexology, nasal sprays, and electrostatically charged nasal application products as previously invented by the present inventor.

The value of monitoring airborne contaminants as a means to avoid or at least minimize the symptoms or complications of allergies is obvious. It may also determine if the user had exposure to toxic or hazardous chemicals, bio-chemicals, viruses, bacteria, or any other airborne particulate of conventional or weaponized form.

Alternative methods of monitoring the presence of airborne contaminants include air sampling, wipe sampling, and bulk sampling.

A method for determining airborne contamination without deliberate exposure or personal inconvenience to the sufferer has been realized. U.S. Pat. Nos. 5,468,488 and 5,674,481 both issued to Ashok L. Wahi, and U.S. patent application Ser. No. 10/082978 filed by Ashok L. Wahi, describes a product, which is intended to inhibit airborne contaminants from entering the nasal passages.

B. Information Disclosure Statement

The following United States Patents relate to attraction and repulsion of airborne contaminants. U.S. Pat. No. 5,468,488 describes a method for restricting the flow of airborne contaminants into nasal passages. It involves creating an electrostatic field in an area near human nasal passages. The electrostatic field may repel or attract airborne contaminants or both. The method involves applying a topical application having a plurality of masses of one or more electrostatic materials, and a carrier having the plurality of masses dispersed therein. The masses have an average cross sectional area between about one square millimeter and about 50,000 square millimeters. They have sufficient charge to create an electrostatic field, which will prevent at least some airborne contaminants from passing into human nasal passages. The topical application may be in the form of a solution, a semisolid, a solid, a spray solution or a vaporizable solution.

U.S. Pat. No. 5,674,481 describes a product and method for restricting the flow of airborne contaminants into nasal passages. It involves creating an electrostatic field in an area near the nasal passages. The electrostatic field may either repel or attract airborne contaminants or both. The product may take the form of a plurality of masses of one or more electrostatic materials, the masses have an average cross sectional area between about one square millimeter and about 50,000 square millimeters. The mass has sufficient charge to create an electrostatic field, which will prevent at least some airborne contaminants from passing into nasal passages. There is also a carrier material with the plurality of masses dispersed therein. The product may be a topical solution, a semi solid, a solid, a spray solution or a vaporizable solution. Alternatively, it may be in a form, which includes a substance for the carrier, and, in one preferred embodiment, the substrate would be an adhesive material such as a bandage.

Notwithstanding the prior art, the present invention is neither taught nor rendered obvious thereby.

### III. SUMMARY OF THE INVENTION

The present invention relates to a method for diagnosing one or more airborne contaminants. The method includes:

(1) Applying a nasal topical application product to a user in proximity to human nasal passages.

(2) Subsequent removal of the nasal topical application product from the user.

(3) Submitting the nasal topical applicator product to a diagnostic system.

(4) Determination of the presence or absence of at least one airborne contaminant.

The nasal topical application product, in one embodiment, may consist of a support strip, a plurality of masses of one or more electrostatic polymers, and a carrier on the strip that has said plurality of masses dispersed throughout at least a portion thereof.

In another embodiment, the nasal topical application product may be fluid consisting of electrostatic polymers and the carrier. The carrier may, alternatively, be a gel selected from the group of three-dimensional polymeric matrices of natural polymers, synthetic polymers, copolymers, and mixtures thereof.

### IV. BRIEF DESCRIPTION OF THE DRAWINGS

FIG. 1 depicts schematically the product concept of the previous invention.

FIG. 2 depicts a side partial stylized view of a human, illustrating a typical electrostatic field around a human nasal passage.

FIG. 3 depicts the same stylized human outline as in FIG. 2 but with an artificially created electrostatic field near a persons nose to restrict the flow of airborne contaminants into the nasal passages.

FIG. 4 depicts another alternative present invention embodiment wherein combinations of artificially created electrostatic fields are used.

FIG. 5 depicts a mild artificially created electrostatic field.

FIG. 6 depicts the present invention method in a flow diagram presentation.

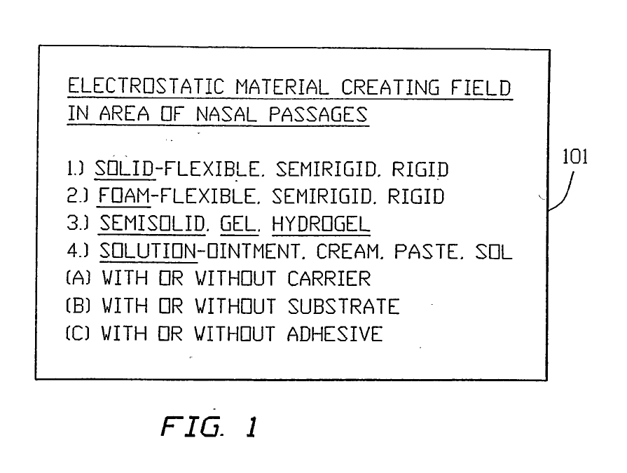

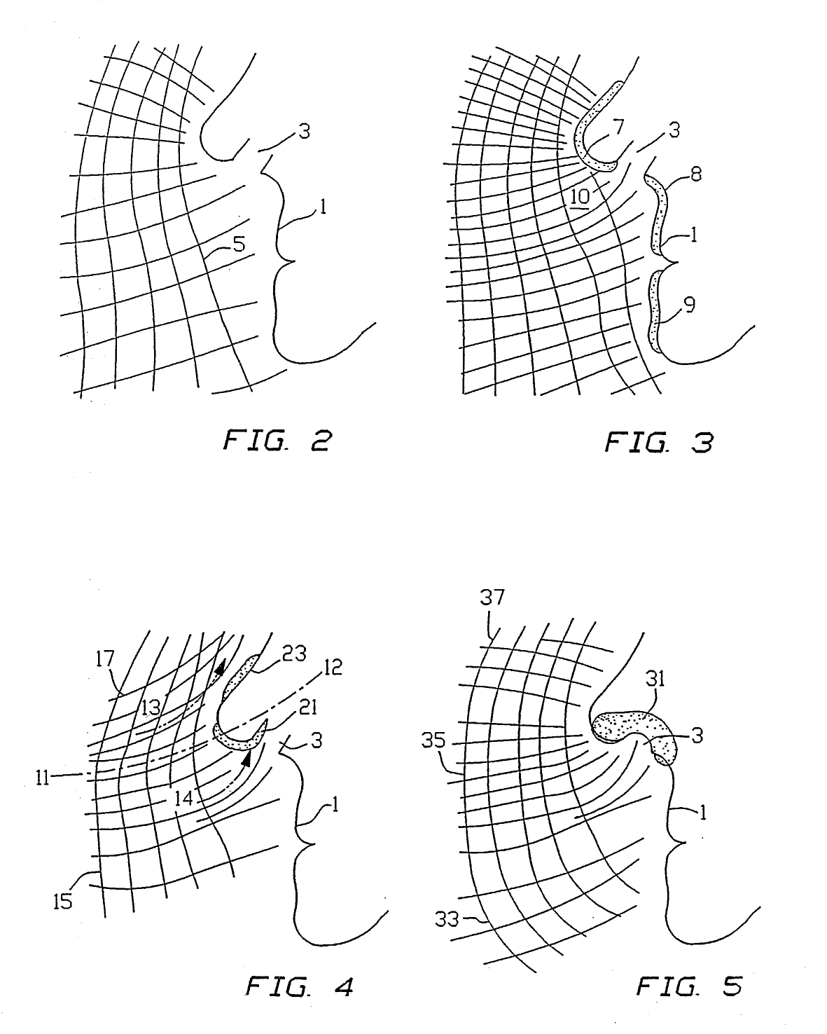

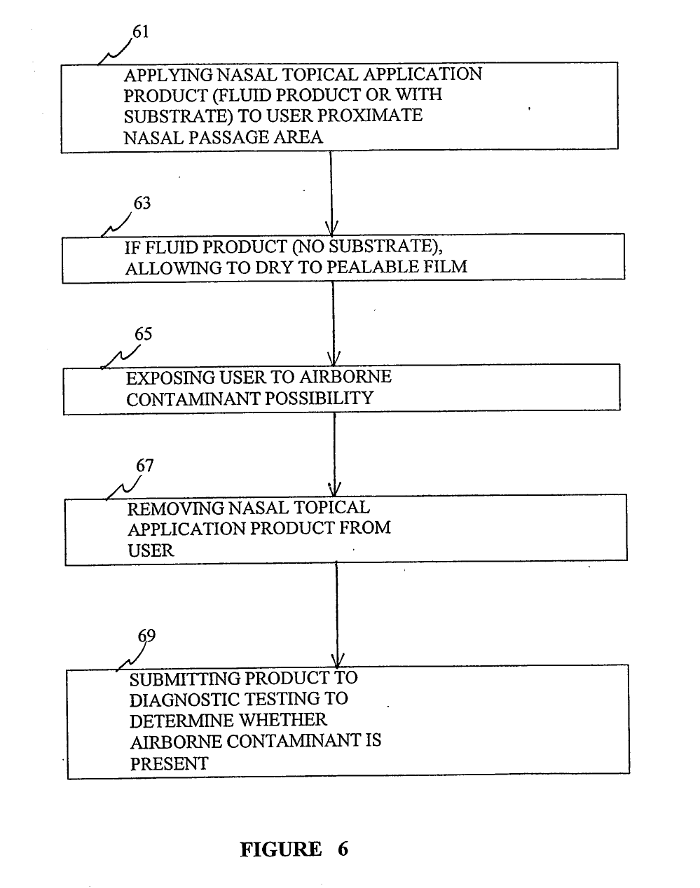

### V. DETAILED DESCRIPTION OF THE PRESENT INVENTION

Before describing the present invention in details, it is understood that this invention is not limited to particular compositions or biological systems. It is also understood that the terminology used herein is for the purpose of describing particular embodiments only, and is not intended to be limiting.

It must be noted that, as used in this specification and the appended claims, the singular forms “a”, “an” and “the” include plural referents unless the content clearly indicates otherwise. For example: reference to “an analyte” includes a mixture of two or more such analytes; reference to “an electrochemically active species” includes two or more such species; and the like.

All publications, patents and patent applications cited herein, whether supra or infra, are hereby incorporated by reference in their entirety.

Unless defined otherwise, all technical and scientific terms used herein have the same meaning as commonly understood by one of ordinary skill in the art to which the invention pertains. Although any methods and materials similar or equivalent to those described herein can be used in the practice of the present invention. The preferred materials and methods are described herein.

In describing and claiming the present invention, the following terminology will be used in accordance with the definitions set out below.

A. Definitions

The terms “analyte” and “target analyte” are used herein to denote any physiological analyte of interest that is a specific substance or component that is being detected and/or measured in a chemical, physical, enzymatic, or optical analysis. A detectable signal (e.g., a chemical signal or electrochemical signal) can be obtained, either directly or indirectly, from such an analyte or derivates thereof. Furthermore, the terms “analyte”, “substance”, “contaminants”, “airborne contaminants”, or “potential contaminants” are used interchangeably herein; are intended to have the same meaning; and therefore include any substance of interest. In preferred embodiments, the analyte is a physiological analyte of interest, such as: pollen, mold spores, dust, dust mites, or other particulate matter or chemicals, some of which have been known to cause allergic reactions, including coughing, sneezing, sinus problems, headaches, or hives. The analyte may also be a toxic or hazardous chemical, bio-chemicals, viruses, bacteria, or any other airborne particulate of either conventional or weaponized form.

The terms “airborne system” or “environmental system” are used herein to denote any physiological environment of interest that a specific subject may be exposed to, whether in the home environment, work environment, educational environment, exercise environment, daily routines, unusual or non-routine travel, or at any other time that may be of interest to the researcher. Furthermore, the terms “airborne system”, “environmental system”, and “ambient system” are used interchangeably herein, and are intended to have the same meaning, and thus include any environment of interest.

A “sampling device” or “sampling system” refers to any device for obtaining a sample from an environmental system for the purpose of determining the concentration of an analyte of interest. As used herein, the term “sampling” means invasive, minimally invasive or noninvasive extraction of a substance from the environmental system, generally across a membrane such as skin, nasal product, or topical application. The membrane can be natural or artificial. It can be of plant or animal nature, such as natural or artificial skin, blood vessel tissue, intestinal tissue, and the like. The nasal application products can be topical solutions, semisolids, spray solutions and vaporizable solutions. Topical applications can be in the form of ointments, pastes, creams and gels. Typically, the sampling device is in operative contact with a “reservoir,” or “collection reservoir,” wherein the sampling means are used for extracting the analyte from the environmental system into the reservoir. Examples of minimally invasive and noninvasive sampling techniques include applying the nasal topical application product to a user in proximity to the human nasal passages; subsequent removal of the nasal topical application product from the user; and submission of nasal topical application product to a diagnostic system.

The term “subject” encompasses any warm-blooded animal, particularly including mammals, humans (as will be typical) and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “continual measurement” means a series of two or more measurements obtained from a particular environmental system. Measurements are obtained using multiple applications in operative contact with the environmental system over the prescribed time period. The term thus includes “continuous measurements.”

The term “transdermal,” as used herein, includes both transdermal and transmucosal techniques, i.e., extraction of a target analyte across skin or mucosal tissue. Aspects of the invention, which are described herein in the context of “transdermal,” unless otherwise specified, are meant to apply to both transdermal and transmucosal techniques.

An object may be described as “topical” if it is either part of or applied to a localized area of the body, or to the surface of a body part.

The terms “transdermal extraction,” “transdermally extracted,” “topical extraction,” “topically extracted,” “extraction” or “extracted”, means any noninvasive, or at least minimally invasive sampling method, which entails extracting and/or transporting an analyte from beneath a tissue surface across skin or mucosal tissue. The term includes applying a nasal topical application product to a user in proximity to human nasal passages; subsequently removing the nasal topical application product from the user; and submitting said nasal topical application product to a diagnostic system.

The term “iontropic” intends a method for attracting substances towards tissue by way of creating an electrostatic field in the desired area. This method is taught in the patents for electrostatically charged nasal topical application products as described in U.S. Pat. No. 5,468,488, U.S. Pat. No. 5,674,481, and Pending U.S. patent application Ser. No. 10/082,978, which utilize electrostatic/anti-stat properties for attracting contaminants to be captured by the topical product.

The term “electrostatic” describes that which pertains to electric forces or charges in a state of rest. An object may be considered “electrostatically charged”, and therefore called an “electrostatic material” or an “electrostatically charged mass”, if it is in the condition of having such a stationary charge. The “electrostatic field” is the region surrounding this charge and in which another charge experiences a force. The “electrostatic charge density” refers to the quantity of static electric force, either per unit area or per unit volume.

The term “active” refers to a chemical agent capable of functioning. It may be considered an “electrostatic active” if it carries an electrostatic charge.

The term “carrier” is used to describe any suitable containment means for containing a sample extracted from an environmental system. Possible carriers include, but are not limited to, a support strip that carries a plurality of masses of one or more electrostatic polymers, natural polymers, synthetic polymers, copolymers and mixtures thereof.

The term “physiological effect” encompasses effects produced in the subject that achieve the intended purpose of a therapy. In preferred embodiments, a physiological effect means that the symptoms of the subject being treated are prevented or alleviated. For example, the subject is asymptomatic with respect to prolonged periods without coughing, sneezing, sinus problems, headaches, hives or any other manifestations of allergic reaction(s).

The term “substrate” refers to base or carrier material over which the application product can be applied.

B. Exemplary Embodiments of Sampling Systems

The present invention relates to electrostatically charged materials, support strips, carriers, and other components useful in a sampling device for transdermally extracting and measuring the concentration of a target analyte present in an environmental system.

The analyte can be any specific substance or component that one is desirous of detecting and/or measuring in a chemical, physical, enzymatic, or optical analysis. Such analytes include, but are not limited to, amino acids, enzyme substrates or products indicating a disease state or condition, other markers of disease states or conditions, drugs of abuse, therapeutic and/or pharmacological agents (e.g., theophylline, anti-HIV drugs, lithium, anti-epileptic drugs, cyclosporin, chemotherapeutics), electrolytes, physiological analytes of interest (e.g., urate/uric acid, carbonate, calcium, potassium, sodium, chloride, bicarbonate (CO.sub.2), glucose, urea (blood urea nitrogen), lactate/lactic acid, hydroxybutyrate, cholesterol, triglycerides, creatine, creatinine, insulin, hematocrit, and hemoglobin), blood gases (carbon dioxide, oxygen, pH), lipids, heavy metals (e.g., lead, copper), and in preferred embodiments, the analyte is a physiological analyte of interest. For example: pollen, mold spores, dust, dust mites, or other particulate matter or chemicals, some of which have been known to cause allergic reactions, such as coughing, sneezing, sinus problems, headaches, or hives. The analyte may also be a toxic or hazardous chemical, bio-chemicals, viruses, bacteria, or any other airborne particulate of either conventional or weaponized form.

In like manner, a number of other analyte-specific enzyme systems can be used in the invention, as they operate on similar general techniques.

More specifically, a non-invasive contaminant-monitoring (sampling) device is used to measure changes in contaminant levels in a subject over a wide range of contamination concentrations. The sampling method is based on transdermal contaminant-extraction. The device can be repeatedly in contact with the environmental system to obtain analyte samples in order to measure analyte concentration at various selected intervals.

Sampling is carried out repeatedly by noninvasive extricating of potential contaminants from the environment by topical extraction. More particularly, an electrostatically charged topical product is applied to a skin surface of a subject. When the product is applied, ions or charged molecules attract other oppositely charged molecules or particles such as contaminants from the subjects' environment, which are drawn into a carrier insert placed on the surface of the skin.

The nasal application product may be topical solution, semisolid, spray solution or vaporizable solution.

Topical applications may be in the form of ointments, pastes, creams and gels.

The carrier of the nasal application product of the present invention may be selected from the group consisting of dilutants, volatile spray carriers, lotions, solvents, gels and hydro-gels.

In some embodiments, substrates, e.g., bandage type substrates, with adhesive on one side and the product polymer(s) and carrier on the opposite side, may be employed.

In other embodiments a fluid dried to film is removable by peeling for testing.

The carrier may further contain an enzyme that catalyzes a reaction of a target contaminant.

When the reaction is complete, the process can be repeated and subsequent measurements obtained. More specifically, when the electrostatically charged topical product is again applied, contaminants are drawn through the air into the carrier, and the reaction is catalyzed. These sampling (extraction) and sensing operations can be integrated.

In one embodiment of the present invention, the nasal topical application product is made up of a support strip, a plurality of masses of one or more electrostatic polymers, and a carrier on the strip that has the plurality of masses dispersed through out at least a portion thereof.

In another embodiment, the nasal topical application product is a fluid product that includes electrostatic polymers and the carrier. The carrier may, alternatively, be a gel selected from the group consisting of three-dimensional polymeric matrices of natural polymers, synthetic polymers, copolymers, and mixtures thereof.

C. General Methods

The invention relates to a method for diagnosing an airborne contaminant. This is done by sampling analytes present in an environmental system, typically a physiologically active material that can attract analytes entering the subject through the nasal passages. The method entails two general steps, a sampling step and a determination step. The sampling step can be generalized as follows. Small sampling particles are electrostatically attracted into a carrier that has been applied on an exposed surface in the target environment. The attraction of these particles is sufficient to bond with carriers that allow a quantity of an analyte of interest to be collected from the environment and deposited on the topical product that has been applied to the subject's skin.

Step A: Obtaining a Sample

The sampling particles typically, but not necessarily, comprise an allergy causing contaminant-material. The material may cause allergic reactions such as coughing, sneezing, sinus problems, headaches, or hives. The sampling particles can be comprised of common materials such as pollen, mold spores, dust, dust mites, or other particulate matter or chemicals.

The sample may be collected from the target surface in a number of ways.

STEP A-1: Apply electrostatically charged material.

Apply a nasal topical application product to a user in proximity to their human nasal passages, e.g., above the upper lip or lower nose or cheek area. This product may consist of a support strip, a plurality of masses of one or more electrostatic polymers.

The nasal application product may or may not be a fluid that will dry to a film upon exposure to air.

This may be a gel-based carrier selected from the group consisting of three-dimensional polymeric matrixes of natural polymers, synthetic polymers, copolymers and mixtures thereof.

STEP A-2: Expose subject to test environment.

The user and product are exposed to the potential airborne contaminant(s) for collection of some of the contaminants by the product.

STEP A-3: Subsequently remove product from user.

If the nasal topical application product is in fluid form, that is, with no substrate, it is permitted to dry to form a pealable film before being removed. It is not necessarily removed as soon as it dries, but must be removed after exposure to possible airborne contaminants.

Remove the nasal topical application product, or the resulting film, from the user.

Step B: Determine the Contents

Submit the nasal topical application product to a diagnostic system to determine whether or not the user has experienced exposure to one or more airborne contaminants. The particular diagnostic methods or tests used to determine the presence or absence of a contaminant on the products described may be any known or available test. For example, numerous tests are well known for determining the presence of anthrax, mustard gas, specific carcinogens, viruses, harmful bacteria, etc.

These are well within the skill of the artisan and are not defined in detail herein. Thus, the diagnostics steps involved in the present invention are a matter of choice by the user.

1. A method for diagnosing an airborne contaminant, which comprises:
(a) Applying a nasal topical application product to a user in proximity to the human nasal passages; and,
(b) Subsequently removing the nasal topical application product from the user and submitting nasal topical application product to a diagnostic system to determine the presence or absence of at least one airborne contaminant; the nasal topical application product to be support strip, a plurality of masses of one or more electrostatic polymers, and a carrier on said strip that has said plurality of masses dispersed through at least a portion thereof.

2. The method of claim 1 wherein said carrier is a gel based carrier selected from the group consisting of 3 dimensional polymeric matrixes of natural polymers, synthetic polymers, copolymers and mixtures thereof.

3. The method for diagnosing an airborne contaminant, which comprises:
(a) Applying a nasal topical application product to a user in proximity to the human nasal passages; and,
(b) Subsequently removing the nasal topical application product from the user and submitting said nasal topical application product to a diagnostic system to determine the presence or absence of at least one airborne contaminant; the nasal topical application product consists of a support strip, a plurality of masses of one or more electrostatic polymers, and all of the above weight percentages being based on the total weight of said plurality of masses of one or more electrostatic polymers and the topical carrier.

4. A method for diagnosing an airborne contaminant, which comprises:
(a) Applying a fluid nasal topical application product to a user in proximity to the human nasal passages, said fluid nasal application product being a product that will dry to a film upon exposure to air;
(b) Allowing said fluid nasal topical application product to dry to film,
(c) Removing the nasal topical application product film from the user and submitting said nasal topical application product film to a diagnostic system to determine the presence or absence of at least one airborne contaminant; said nasal topical application product consisting of a plurality of masses of one or more electrostatic polymers, and a topic carrier in proximity to the nasal passages.
